(19) United States
(12) Patent Application Publication
(10) Pub. No.: US 2025/0213460 A1
(43) Pub. Date: Jul. 3, 2025

(54) NATURAL DERIVED FILM FORMER FOR COSMETIC APPLICATIONS

(71) Applicant: MOMENTIVE PERFORMANCE MATERIALS INC., Niskayuna, NY (US)

(72) Inventors: Roy Uwe ROJAS WAHL, Saddle River, NJ (US); Peng LI, Niskayuna, NY (US); Chris MAHONEY, Park Ridge, NJ (US); Sigfredo GONZALEZ, Danbury, CT (US); Amar PAWAR, Briarcliff Manor, NY (US); Benjamin FALK, Niskayuna, NY (US); Hao Shen, Verona, NJ (US)

(21) Appl. No.: 19/003,382

(22) Filed: Dec. 27, 2024

Related U.S. Application Data
(60) Provisional application No. 63/615,845, filed on Dec. 29, 2023.

(51) Int. Cl. A61K 8/9789; A61Q 19/00; C08H 99/00

(52) U.S. Cl. CPC A61K 8/9789; A61Q 19/00; C08H 99/00; A61K 2800/10

(57) ABSTRACT

The present disclosure provides a natural compound derived from Dammar gum that when formulated into a composition for personal care, delivers color transfer resistance, even when exposed to oil, water, and/or food. The present disclosure also provides methods of making the compound derived from Dammar gum and compositions comprising the compound derived from Dammar gum. The compound derived from Dammar gum can be combined with components typically used in personal care to provide personal care formulations.

### CROSS REFERENCE TO RELATED APPLICATIONS

This application claims priority to U.S. Provisional Patent Application No. 63/615,845, filed Dec. 29, 2023, which is incorporated herein by reference in its entirety.

### FIELD

The present disclosure provides a natural compound derived from Dammar gum that when formulated into a composition for personal care, delivers color transfer resistance, even when exposed to oil, water, and/or food. The present disclosure also provides methods of making the compound derived from Dammar gum and compositions comprising the compound derived from Dammar gum. The compound derived from Dammar gum can be combined with components typically used in personal care to provide personal care formulations.

### BACKGROUND

There exists a need in the market for non-silicone, non-acrylate, 100% natural film formers for use in cosmetic applications, especially in lip-color and lipstick, providing resistance to (a) general color transfer, (b) color transfer when exposed to food oils, and/or (c) color transfer when exposed to beverages containing water.

Natural resins of plant or animal origin constitute an advantageous product class by virtue of their special properties linked to nature. However, such resins of natural origin are rarely used in cosmetic compositions because they are generally insoluble in water, a ubiquitous solvent used in the cosmetic industry. Attempts have been made to adapt these natural resins to a form that is suitable for use with water. For example, U.S. Pat. No. 5,720,943 discloses the preparation of an aqueous dispersion of natural resins used in a cosmetic composition. These aqueous dispersions of natural resins, including Dammar gum resin, demonstrated good film-forming properties.

The present disclosure provides an improved approach to the preparation of a natural resin for use in personal care formulations, in particular for lipsticks and lip glosses. In the present disclosure, a polymeric fraction of a Dammar gum is prepared and the compound derived from the Dammar gum can be used in personal care formulations which are not limited to aqueous systems or dispersions. The derived Dammar gum personal care formulations provide improved transfer resistance, water resistance, and oil resistance.

### SUMMARY

The present disclosure provides a compound derived from a Dammar gum comprising: at least about 80 mol % saturated branched hydrocarbons; between about 5 mol % and about 20 mol % unsaturated hydrocarbons; and less than about 1 mol % of carbonyl carbons, wherein the carbonyl carbons comprise aldehydes, esters, and acids.

In some aspects, the compound derived from a Dammar gum is further characterized as having an average number molecular weight (Mn) of about 2000 g/mol to about 5000 g/mol as measured by gel permeation chromatography (GPC).

In some aspects, the compound derived from a Dammar gum is further characterized as having a Mn of about 2500 g/mol to about 3700 g/mol as measured by GPC.

In some aspects, the compound derived from a Dammar gum is further characterized as having a Mn of about 3418 g/mol as measured by GPC.

In some aspects, the compound derived from a Dammar gum is further characterized by GPC:
(a) as having a Mn of about 3153 g/mol and a polydispersity of about 1.5 using polystyrene 500-1,000,000 Da standards, in methylene chloride solvent, with two PHENOGEL 5 μm linear (2) columns (dimensions: 300 mm×7.8 mm) and one PHENOGEL 5 μm linear (2) guard column (dimensions: 50 mm×7.8 mm); or
(b) as having a Mn of about 3418 g/mol and a polydispersity of about 1.3 using polystyrene 500-1,000,000 Da standards, in methylene chloride solvent, with two PLgel 5 μm MIXED-D columns (dimensions: 300 mm×7.5 mm), and one PLgel 5 μm MIXED guard column (dimensions: 50 mm×7.8 mm); or
(c) as having a Mn of about 612 and a polydispersity of about 5.7 using polystyrene 500-1,000,000 Da standards, in toluene solvent, with two PHENOGEL 5 μm linear (2) columns (dimensions: 300 mm×7.8 mm) and one PHENOGEL 5 μm linear (2) guard column (dimensions: 50 mm×7.8 mm).

In some aspects, the compound derived from a Dammar gum is further characterized as having a highest rate of decomposition of about 350° C. to about 450° C. as measured by thermal gravimetric analysis (TGA).

In some aspects, the compound derived from a Dammar gum is further characterized as having a highest rate of decomposition of about 395° C. as determined by TGA.

In some aspects, the compound derived from a Dammar gum is further characterized as having a IR spectrum with peaks at 3439, 2956, 2931, and 2869 cm⁻¹ (=4 cm⁻¹).

In some aspects, the compound derived from a Dammar gum is further characterized as having a IR spectrum with peaks at 3439, 2956, 2931, 2869, 1708, 1628, 1464, and 1384 cm⁻¹ (±4 cm⁻¹).

The present disclosure also provides a composition comprising the compound derived from a Dammar gum and a plasticizer.

In some aspects, in the composition comprising the compound derived from a Dammar gum and a plasticizer, the plasticizer is selected from the group consisting of a trimethylpentanediol/adipic acid/glycerin crosspolymer, capryloyl glycerin/sebacic acid copolymer, Brassica campestris/Aleurites fordii oil copolymer, PPG-12/SMDI copolymer, polyisobutylene, polyisobutene 1200, isocetyl salicylate, polyhydroxystearic acid, and dicaprylyl/capryl sebacate.

In some aspects, in the composition comprising the compound derived from a Dammar gum and a plasticizer, the plasticizer is selected from the group consisting of LEXOREZ 200 MB, VELLAPLEX MB, GLOSSAMER L6600, ADEKA NOL OU-1, LIPFEEL Natural MB, polyisobutylene, polyisobutene 1200, DERMOL ICSA, KESTER WAX K-60P, and HALLGREEN CCS.

The present disclosure also provides a personal care formulation comprising the compound derived from a Dammar gum or the composition comprising the compound derived from a Dammar gum and a plasticizer.

In some aspects, the personal care formulation forms a film when applied to skin or lips.

In some aspects, the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a lip gloss, a lip color, a liquid lip, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

### BRIEF DESCRIPTION OF THE DRAWINGS

FIG. 1 shows a gel permeation chromatography (GPC) spectrum of the compound derived from Dammar gum of Example 1. The GPC spectrum shows a molecular weight at the peak (Mp) value of 4396 g/mol; a Mn value of 3418 g/mol; a weight average molecular weight (Mw) value of 4371 g/mol; a Z-average molar mass (Mz) value of 555.1 g/mol; and a polydispersity index (PDI) of 1.27882 for the compound derived from Dammar gum of Example 1.

FIG. 2 shows a GPC spectrum of two commercially available Dammar gums. “Damar-EZ Nexira,” a commercially available Dammar gum with tree bark removed (DAMAR-EZ Organic, (Nexira, Rouen, France), shows a Mn value of 318 g/mol; a Mw value of 361 g/mol; and a Mz value of 419 g/mol. “Damar Gum-WB,” a commercially available Dammar gum from Indonesia (William Bernstein Company, New York, NY), shows a Mn value of 295 g/mol; a Mw value of 33 g/mol; and a Mz value of 402.

FIG. 3 shows an Infrared (IR) spectrum of the compound derived from a Dammar gum of Example 1.

FIG. 4 shows IR spectra for DAMAR-EZ Organic (“Damar-EZ Nexira”) and for a commercially available Dammar gum from Indonesia (“Damar Gum-WB”).

FIG. 5 shows the transfer resistance (“TR”), water resistance (“WR”), and oil resistance (“OR”) results of the personal care formulations of Example 5 for TESTs 1-4 in TABLE 3. TEST 2 shows the results for a compound derived from a Dammar gum.

FIG. 6 shows the transfer resistance (“TR”), water resistance (“WR”), and oil resistance (“OR”) results of the personal care formulations of Example 5 for TESTs 5-7 in TABLE 3. Test 7 shows the results for a compound derived from a Dammar gum.

FIG. 7 shows the transfer resistance (“TR”), water resistance (“WR”), and oil resistance (“OR”) results of the personal care formulation prepared from the compound derived from a Dammar gum of Example 2.

FIG. 8 shows the transfer resistance (“TR”), water resistance (“WR”), and oil resistance (“OR”) results of the personal care formulation prepared from the compound derived from a Dammar gum of Example 3.

FIG. 9 shows the transfer resistance (“TR”), water resistance (“WR”), and oil resistance (“OR”) results of the personal care formulation prepared from the compound derived from a Dammar gum of Example 4.

### DETAILED DESCRIPTION

### I. Definitions

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the region of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

All ranges disclosed herein are inclusive of the endpoints, and the endpoints are independently combinable with each other. Each range disclosed herein constitutes a disclosure of any point or sub-range lying within the disclosed range. For example, the range from X to Y, is inclusive of X and Y. And, the range between X and Y, is inclusive of X and Y.

The term “hydrocarbon,” as used herein, refers to any chemical structure containing carbon atoms and hydrogen atoms. The hydrocarbon can be linear, branched, or cyclic.

The term “saturated hydrocarbon,” as used herein, refers a hydrocarbon with no unsaturated carbon atoms or hydrocarbon atoms. The saturated hydrocarbon can be linear, branched, or cyclic. For example, the hydrocarbon can be a substituted or an unsubstituted alkane and/or a substituted or an unsubstituted cycloalkane. The saturated hydrocarbons can have a general formula of an unsubstituted alkane as CₙH₂ₙ₊₂ where n can be 2-2000, 2-1000, 2-500, 2-200, 2-100, 2-80, or 2-50. In some aspects, one or more hydrogens on the alkane or the cycloalkanes can be further substituted with other functional groups such as but not limited to, halogen (including chloro, bromo, iodo, and fluoro), carboxylic acid (—COOH), hydroxyl (—OH), and amines. Examples of saturated hydrocarbons include ethane, propane, butane, pentane, methyl butane, and hexane.

The term “unsaturated hydrocarbon,” as used herein, refers to a hydrocarbon with unsaturated carbon or hydrocarbon with at least one double and/or at least one triple bond between adjacent carbon atoms. The unsaturated hydrocarbon can be linear, branched, or cyclic (aromatic or non-aromatic). For example, the hydrocarbon can be olefinic, acetylenic, non-aromatic such as cyclohexene, aromatic group or a substituted unsaturated hydrocarbon such as, but not limited to, halogenated unsaturated hydrocarbon. The hydrocarbons with at least one double bond can be called alkenes and can have a general formula of an unsubstituted alkene as CₙH₂ₙ where n can be 2-2000, 2-1000, 2-500, 2-200, 2-100, 2-80, or 2-50. In some aspects, one or more hydrogens on the alkene can be further substituted with other functional groups such as but not limited to, halogen (including chloro, bromo, iodo, and fluoro), carboxylic acid (—COOH), hydroxyl (—OH), and amine. The unsaturated hydrocarbons include all the isomeric forms of unsaturation, such as, but not limited to, cis and trans isomers, E and Z isomers, and positional isomers.

The term “branched” refers to an open chain of atoms with one or more side chains attached to it.

The term “alkyl” means any monovalent, saturated straight chain or branched chain hydrocarbon group; the term “alkenyl” means any monovalent straight chain or branched chain hydrocarbon group containing one or more carbon-carbon double bonds where the site of attachment of the group can be either at a carbon-carbon double bond or elsewhere therein; and, the term “alkynyl” means any monovalent straight chain or branched chain hydrocarbon group containing one or more carbon-carbon triple bonds and, optionally, one or more carbon-carbon double bonds, where the site of attachment of the group can be either at a carbon-carbon triple bond, a carbon-carbon double bond or elsewhere therein.

Representative examples of alkyls include methyl, ethyl, propyl and isobutyl. Examples of alkenyls include vinyl, propenyl, allyl, methallyl, ethylidenyl norbornane, ethylidene norbornyl, ethylidenyl norbornene, and ethylidene norbornenyl. Examples of alkynyls include acetylenyl, propargyl, and methylacetylenyl.

The term “cycloalkyl” means any monovalent cyclic aliphatic hydrocarbon group; the term “cycloalkenyl” means any monovalent cyclic aliphatic hydrocarbon group containing one or more carbon-carbon double bonds where the site of attachment of the group can be either at a carbon-carbon double bond or elsewhere therein; and, the term “cycloalkynyl” means any monovalent cyclic aliphatic hydrocarbon group containing one or more carbon-carbon triple bonds and, optionally, one or more carbon-carbon double bonds, where the site of attachment of the group can be either at a carbon-carbon triple bond, a carbon-carbon double bond or elsewhere therein.

Representative examples of cycloalkyl include cyclopentyl, cyclobutyl, cyclopentyl, cycloheptyl, cyclooctyl. Examples of cyloalkenyl include cyclopentenyl, cycloheptenyl, and cyclooctatrienyl. An example of cycloalkynyl is cycloheptynyl.

The terms “cycloalkyl”, “cycloalkenyl”, and “cycloalkynyl” include bicyclic, tricyclic and higher cyclic structures as well as the aforementioned cyclic structures further substituted with alkyl, alkenyl, and/or alkynyl groups. Representative examples include norbornyl, norbornenyl, ethylnorbornyl, ethylnorbornenyl, cyclohexyl, ethylcyclohexyl, ethylcyclohexenyl, cyclohexylcyclohexyl, and cyclododecatrienyl.

The term “aryl” includes any aromatic hydrocarbon from which one hydrogen atom has been removed; “aralkyl” includes any of the aforementioned alkyl groups in which one or more hydrogen atoms have been substituted by the same number of like and/or different aryl (as defined herein) substituents; and “arenyl” includes any of the aforementioned aryl groups in which one or more hydrogen atoms have been substituted by the same number of like and/or different alkyl (as defined herein) substituents. Specific, non-limiting examples of aryl groups include phenyl and naphthalenyl. Specific, non-limiting examples of aralkyl groups include benzyl and phenethyl. Specific, non-limiting examples of arenyl groups include tolyl and xylyl.

The term “alkylene” is a divalent saturated aliphatic radical derived from an alkane by removal of two hydrogen atoms. The term “alkenylene” is a divalent unsaturated aliphatic radical derived from an alkene by removal of four hydrogen atoms.

The term “carbonyl compound,” as used herein, refers to a —C(═O) group. Various aspects of the disclosure are described in greater detail below.

### II. Compounds

Dammar gum is the general name for a group of natural resins that originate from trees of the Dipterocarpaceae family which are found in India and in Southeast Asia, primarily in Malaya, Indonesia and the East Indies. Dammar gum is a triterpenoid resin, containing a large number of triterpenes and their oxidation products. Many of the triterpenes are low molecular weight compounds which easily oxidize and photoxidize. The triterpenes in Dammar gum include dammarane, dammarenolic acid, oleanane, and oleanonic acid. Typically, naturally available Dammar gums contain dirt, insoluble material, and/or other gelatinous, non-resinous particles. In order to remove this additional matter, it is necessary to process the gums.

In one aspect, the present disclosure provides a compound derived from a Dammar gum comprising: at least about 80 mol % saturated branched hydrocarbons; between about 5 mol % and about 20 mol % unsaturated hydrocarbons; and less than about 1 mol % of carbonyl carbons, wherein the carbonyl carbons comprise aldehydes, esters, and acids.

In some aspects, the compound derived from a Dammar gum comprises at least 80 mol % saturated branched hydrocarbons. In some aspects, the compound derived from a Dammar gum comprises at least about 85 mol % saturated branched hydrocarbons. In some aspects, the compound derived from a Dammar gum comprises at least about 90 mol % saturated branched hydrocarbons. In some aspects, the compound derived from a Dammar gum comprises at least about 95 mol % saturated branched hydrocarbons.

In some aspects, the compound derived from a Dammar gum comprises between about 80 mol % and about 95 mol % saturated branched hydrocarbons. In some aspects, the compound derived from a Dammar gum comprises between about 80 mol % and about 95 mol %, between about 80 mol % and about 90 mol %, between about 80 mol % and about 85 mol %, between about 85 mol % and about 95 mol %, between about 85 mol % and about 90 mol %, or between about 90 mol % and about 95 mol % saturated branched hydrocarbons.

In some aspects, the compound derived from a Dammar gum comprises about 80 mol %, about 85 mol %, about 90 mol %, or about 95 mol % saturated branched hydrocarbons.

In some aspects, the compound derived from a Dammar gum comprises between about 80 mol % and about 95 mol %, between about 80 mol % and about 90 mol %, between about 80 mol % and about 85 mol %, between about 85 mol % and about 95 mol %, between about 85 mol % and about 90 mol %, or between about 90 mol % and about 95 mol % of an optionally substituted branched alkyl group having from about 2 to about 2000 carbon atoms and optionally at least one heteroatom.

In some aspects, the compound derived from a Dammar gum comprises between about 80 mol % and about 95 mol % of an optionally substituted branched alkyl group having from about 2 to about 2000 carbons atoms, from about 2 to about 1000 carbon atoms, from about 2 to about 500 carbon atoms, from about 2 to about 100 carbon atoms, from about 100 to about 2000 carbon atoms, from about 100 to about 1000 carbon atoms, from about 100 to 500 carbon atoms, from about 500 to about 2000 carbon atoms, from about 500 to about 1000 carbon atoms, or from about 1000 to about 2000 carbon atoms and optionally at least one heteroatom.

In some aspects, the compound derived from a Dammar gum comprises between about 80 mol % and about 95 mol %, between about 80 mol % and about 90 mol %, between about 80 mol % and about 85 mol %, between about 85 mol % and about 95 mol %, between about 85 mol % and about 90 mol %, or between about 90 mol % and about 95 mol % of an optionally substituted branched cycloalkyl group having from about 3 to about 2000 carbon atoms and optionally at least one heteroatom.

In some aspects, the compound derived from a Dammar gum comprises between about 80 mol % and about 95 mol % of an optionally substituted branched cycloalkyl group having from about 3 to about 2000 carbons atoms, from about 3 to about 1000 carbon atoms, from about 3 to about 500 carbon atoms, from about 3 to about 100 carbon atoms, from about 100 to about 2000 carbon atoms, from about 100 to about 1000 carbon atoms, from about 100 to 500 carbon atoms, from about 500 to about 2000 carbon atoms, from about 500 to about 1000 carbon atoms, or from about 1000 to about 2000 carbon atoms and optionally at least one heteroatom.

In some aspects, the compound derived from a Dammar gum comprises between about 5 mol % and about 20 mol % unsaturated hydrocarbons. In some aspects, the compound derived from a Dammar gum comprises between about 5 mol % and about 20 mol %, between about 5 mol % and about 15 mol %, between about 5 mol % and about 10 mol %, between about 10 mol % and about 20 mol %, between about 10 mol % and about 15 mol %, or between about 15 mol % and about 20 mol % of unsaturated branched hydrocarbons.

In some aspects, the compound derived from a Dammar gum comprises about 5 mol %, about 10 mol %, about 15 mol %, or about 20 mol % of unsaturated branched hydrocarbons.

In some aspects, the compound derived from a Dammar gum comprises between about 5 mol % and about 20 mol %, between about 5 mol % and about 15 mol %, between about 5 mol % and about 10 mol %, between about 10 mol % and about 20 mol %, between about 10 mol % and about 15 mol %, or between about 15 mol % and about 20 mol % of an optionally substituted branched alkenyl group having from about 2 to about 2000 carbon atoms and optionally at least one heteroatom.

In some aspects, the compound derived from a Dammar gum comprises between about 5 mol % and about 20 mol % of an optionally substituted branched alkenyl group having from about 2 to about 2000 carbons atoms, from about 2 to about 1000 carbon atoms, from about 2 to about 500 carbon atoms, from about 2 to about 100 carbon atoms, from about 100 to about 2000 carbon atoms, from about 100 to about 1000 carbon atoms, from about 100 to 500 carbon atoms, from about 500 to about 2000 carbon atoms, from about 500 to about 1000 carbon atoms, or from about 1000 to about 2000 carbon atoms and optionally at least one heteroatom.

In some aspects, the compound derived from a Dammar gum comprises between about 5 mol % and about 20 mol %, between about 5 mol % and about 15 mol %, between about 5 mol % and about 10 mol %, between about 10 mol % and about 20 mol %, between about 10 mol % and about 15 mol %, or between about 15 mol % and about 20 mol % of an optionally substituted branched aryl group having from about 6 to about 30 carbon atoms and optionally at least one heteroatom.

In some aspects, the compound derived from a Dammar gum comprises between about 5 mol % and about 20 mol %, between about 5 mol % and about 15 mol %, between about 5 mol % and about 10 mol %, between about 10 mol % and about 20 mol %, between about 10 mol % and about 15 mol %, or between about 15 mol % and about 20 mol % an optionally substituted aralkyl group having from about 7 to about 2000 carbon atoms and optionally at least one heteroatom.

In some aspects, the compound derived from a Dammar gum comprises between about 5 mol % and about 20 mol % of an optionally substituted aralkyl group having from about 7 to about 2000 carbons atoms, from about 7 to about 1000 carbon atoms, from about 7 to about 500 carbon atoms, from about 7 to about 100 carbon atoms, from about 100 to about 2000 carbon atoms, from about 100 to about 1000 carbon atoms, from about 100 to 500 carbon atoms, from about 500 to about 2000 carbon atoms, from about 500 to about 1000 carbon atoms, or from about 1000 to about 2000 carbon atoms and optionally at least one heteroatom.

In some aspects, the compound derived from a Dammar gum comprises less than about 1 mol % of carbonyl compounds. In some aspects, the compound derived from a Dammar gum comprises less than about 1 mol %, less than about 0.5 mol %, or less than about 0.1 mol % of carbonyl compounds.

In some aspects, the compound derived from a Dammar gum comprises less than about 1 mol % of aldehydes. In some aspects, the compound derived from a Dammar gum comprises less than about 1 mol %, less than about 0.5 mol %, or less than about 0.1 mol % of aldehydes.

In some aspects, the compound derived from a Dammar gum comprises less than about 1 mol % of esters. In some aspects, the compound derived from a Dammar gum comprises less than about 1 mol %, less than about 0.5 mol %, or less than about 0.1 mol % of esters.

In some aspects, the compound derived from a Dammar gum comprises less than about 1 mol % of acids. In some aspects, the compound derived from a Dammar gum comprises less than about 1 mol %, less than about 0.5 mol %, or less than about 0.1 mol % of acids.

### III. Characterization

In some aspects, the compound is characterized as having an average number molecular weight (Mn) of about 2000 g/mol to about 5000 g/mol as measured by gel permeation chromatography (GPC). In some aspects, the compound is characterized as having an average number molecular weight (Mn) of about 2000 g/mol to about 5000 g/mol, about 2000 g/mol to about 4500 g/mol, about 2000 g/mol to about 4000 g/mol, about 2000 g/mol to about 3500 g/mol, about 2000 g/mol to about 3000 g/mol, about 2000 g/mol to about 2500 g/mol, about 2500 g/mol to about 5000 g/mol, about 2500 g/mol to about 4500 g/mol, about 2500 g/mol to about 4000 g/mol, about 2500 g/mol to about 3500 g/mol, about 2500 g/mol to about 3000 g/mol, about 3000 g/mol to about 5000 g/mol, about 3500 g/mol to about 5000 g/mol, about 3500 g/mol to about 4500 g/mol, about 3500 g/mol to about 4000 g/mol, about 4000 g/mol to about 5000 g/mol, about 4000 g/mol to about 4500 g/mol, or about 4500 g/mol to about 5000 g/mol as measured by GPC. In some aspects, the compound is characterized as having a Mn of about 2500 g/mol to about 3700 g/mol as measured by GPC.

In some aspects, the compound is characterized as having a Mn of about 2500 g/mol, about 3000 g/mol, about 3500 g/mol, about 4000 g/mol, about 4500 g/mol, or about 5000 g/mol. In some aspects, the compound is characterized as having a Mn of about 3418 g/mol as measured by GPC.

In some aspects, the compound is further characterized by GPC:
(a) as having a Mn of about 3153 g/mol and a polydispersity of about 1.5 using polystyrene 500-1,000,000 Da standards, in methylene chloride solvent, with two PHENOGEL 5 μm linear (2) columns (dimensions: 300 mm×7.8 mm) and one PHENOGEL 5 μm linear (2) guard column (dimensions: 50 mm×7.8 mm); or
(b) as having a Mn of about 3418 g/mol and a polydispersity of about 1.3 using polystyrene 500-1,000,000 Da standards, in methylene chloride solvent, with two PLgel 5 μm MIXED-D columns (dimensions: 300 mm×7.5 mm), and one PLgel 5 μm MIXED guard column (dimensions: 50 mm×7.8 mm); or
(c) as having a Mn of about 612 and a polydispersity of about 5.7 using polystyrene 500-1,000,000 Da standards, in toluene solvent, with two PHENOGEL 5 μm linear (2) columns (dimensions: 300 mm×7.8 mm) and one PHENOGEL 5 μm linear (2) guard column (dimensions: 50 mm×7.8 mm).

In some aspects, the compound is characterized by GPC as having a Mn of about 3153 g/mol and a polydispersity of about 1.5 using polystyrene 500-1,000,000 Da standards, in methylene chloride solvent, with two PHENOGEL 5 μm linear (2) columns (dimensions: 300 mm×7.8 mm) and one PHENOGEL 5 μm linear (2) guard column (dimensions: 50 mm×7.8 mm).

In some aspects, the compound is characterized by GPC as having a Mn of about 3418 g/mol and a polydispersity of about 1.3 using polystyrene 500-1,000,000 Da standards, in methylene chloride solvent, with two PLgel 5 μm MIXED-D columns (dimensions: 300 mm×7.5 mm), and one PLgel 5 μm MIXED guard column (dimensions: 50 mm×7.8 mm).

In some aspects, the compound is characterized by GPC as having a Mn of about 612 and a polydispersity of about 5.7 using polystyrene 500-1,000,000 Da standards, in toluene solvent, with two PHENOGEL 5 μm linear (2) columns (dimensions: 300 mm×7.8 mm) and one PHENOGEL 5 μm linear (2) guard column (dimensions: 50 mm×7.8 mm).

In some aspects, the compound is characterized as having a highest rate of decomposition of about 350° C. to about 450° C. as measured by thermal gravimetric analysis (TGA). In some aspects, the compound is characterized as having a highest rate of decomposition of about 350° C. to about 450° C., about 350° C. to about 425° C., about 350° C. to about 400° C., about 350° C. to about 375° C., about 375° C. to about 450° C., about 375° C. to about 425° C., about 375° C. to about 400° C., about 400° C. to about 450° C., about 400° C. to about 425° C., or about 425° C. to about 450° C. as measured by TGA.

In some aspects, the compound is characterized as having a highest rate of decomposition of about 450° C., about 425° C., about 400° C., about 395° C., about 375° C., or about 350° C. as determined by TGA. In some aspects, the compound is characterized as having a highest rate of decomposition of about 395° C. as determined by TGA.

In some aspects, the compound is characterized as having an Infrared (IR) spectrum with peaks at 3439, 2956, 2931, and 2869 cm⁻¹ (±4 cm⁻¹).

In some aspects, the compound is characterized as having a IR spectrum with peaks at 3439, 2956, 2931, 2869, 1708, 1628, 1464, and 1384 cm⁻¹ (±4 cm⁻¹).

In some aspects, the compound is characterized as having an IR with peaks at 3439, 2956, 2931, 2869, 1708, 1628, 1464, 1384, 1368, 1261, 1107, 1045, 887, and 809 cm⁻¹ (±4 cm⁻¹).

In some aspects, the compound is characterized as having a glass transition temperature (T_{g}) of from about 30° C. to about 100° C., from about 30° C. to about 70° C., from about 30° C. to about 50° C., from about 50° C. to about 100° C., from about 50° C. to about 70° C., or from about 70° C. to about 100° C. In some aspects, the compound is characterized as having a T_{g} of from about 50° C. to about 70° C.

### IV. Compositions

In some aspects, the present disclosure is directed to a composition comprising a compound derived from a Dammar gum and further comprising:
(a) at least one non-volatile oil; or
(b) at least one volatile oil; or
(c) at least one elastomer; or
a combination thereof.

In some aspects, the present disclosure is directed to a composition comprising a compound derived from a Dammar gum and further comprising at least one non-volatile oil, at least one volatile oil, and at least one elastomer.

In some aspects, the present disclosure is directed to a composition comprising a compound derived from a Dammar gum and further comprising at least one volatile oil. The phrase “volatile oil” refers to an oil capable of evaporating at room temperature. In some aspects, the volatile oil is a silicone oil such as decamethylcyclopentasiloxane, polymethylcyclohexasiloxane, and polymethylcyclotetrasiloxane; a linear, branched, or mixture of hydrocarbon oils such as nonane, decane, dodecane, isododecane, isohexadecane, isoparaffin (C₁₀-C₁₄), C₉-C₁₂ alkane, or a combination thereof.

In some aspects, the present disclosure is directed to a composition comprising a compound derived from a Dammar gum and further comprising at least one non-volatile oil. The phrase “non-volatile oil” refers to an oil remaining on a surface, such as skin, at ambient temperature and atmospheric pressure for a plurality of hours, in the absence of friction, and/or having a vapor pressure less than 0.001 mm Hg under these conditions. In some aspects, the non-volatile oil is a mineral or synthetic branched hydrocarbon, a C₂-C₂₄ acid, a C₂-C₂₄ alcohol or polyol, a C₆-C₂₀ fatty acid triglyceride, a vegetable oil, a dialkyl carbonate, a branched and/or unsaturated fatty acid, a branched and/or unsaturated fatty alcohol, a silicone oil, a fluorosilicone oil, a fluorinated oil, or a combination thereof. In some embodiments, the non-volatile oil is selected from the group consisting of ethyl myristate, isopropyl myristate, diisopropyl adipate, and other fatty acids. In some aspects, the non-volatile oil is a plasticizer.

In some aspects, the present disclosure is directed to a composition that does not comprise a volatile alcohol. The term “volatile alcohol” refers to a compound containing at least one hydroxyl group and in which more than 95% by weight of the compound can evaporate in less than one hour at ambient temperature (25° C.) and atmospheric pressure (760 mm Hg) on contact with a keratin material such as the skin or the hair. In some aspects, the volatile alcohol is a lower C₁-C₅ alcohol such as methanol, ethanol, propanol, isopropanol, n-butanol, tert-butanol, and combinations thereof.

In some aspects, the present disclosure is directed to a composition comprising a compound derived from a Dammar gum and a plasticizer. In some aspects, the composition is a personal care composition. The phrase “personal care composition” or “personal care formulation” is used to describe a chemical composition used for the purpose of cleansing, conditioning, grooming, beautifying, or otherwise enhancing the appearance of the human body.

In some aspects, the plasticizer provides additional consistency and stability to the composition. Plasticizers can fix graininess that occurs when solids migrate and agglomerate. Plasticizers can also fix thermal stability issues by increasing melt point, without affecting the overall texture.

In some aspects, the compositions comprise a plasticizer in a weight ratio from about 0.01% to about 80% of the total weight of the composition. In some aspects, the compositions comprise a plasticizer in a weight ratio from about 0.01% to about 80%, from about 0.01% to about 60%, from about 0.01% to about 40%, from about 0.01% to about 20%, from about 0.01% to about 10%, from about 0.01% to about 5%, from about 0.01% to about 1%, from about 1% to about 80%, from about 1% to about 60%, from about 1% to about 40%, from about 1% to about 20%, from about 1% to about 10%, from about 1% to about 5%, from about 5% to about 80%, from about 5% to about 60%, from about 5% to about 40%, from about 5% to about 20%, from about 5% to about 10%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% of the total weight of the composition. In some aspects, the compositions comprise a plasticizer in a weight ratio of about 0.01%, about 1%, about 5%, about 10%, about 20%, about 40%, about 60%, or about 80% of the total weight of the composition.

In some aspects, the plasticizer is selected from the group consisting of cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂₋₁₃ alkyl lactate, C₁₂₋₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂₋₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxystearate, trimethylpentanediol/adipic acid/glycerin crosspolymer, capryloyl glycerin/sebacic acid copolymer, Brassica campestris/Aleurites fordii oil copolymer, PPG-12/SMDI copolymer, polyisobutylene, polyisobutene 1200, polyhydroxystearic acid, and dicaprylyl/capryl sebacate, and combinations thereof.

In some aspects, the plasticizer is selected from the group consisting of a trimethylpentanediol/adipic acid/glycerin crosspolymer, capryloyl glycerin/sebacic acid copolymer, Brassica campestris/Aleurites fordii oil copolymer, PPG-12/SMDI copolymer, polyisobutylene, polyisobutene 1200, isocetyl salicylate, polyhydroxystearic acid, dicaprylyl/capryl sebacate, and combinations thereof.

In some aspects, the plasticizer is selected from the group consisting of LEXOREZ 200 MB, VELLAPLEX MB, GLOSSAMER L6600, ADEKA NOL OU-1, LIPFEEL Natural MB, polyisobutylene, polyisobutene 1200, DERMOL ICSA, KESTER WAX K-60P, HALLGREEN CCS, and combinations thereof.

In some aspects, the present disclosure is directed to a composition comprising the compound derived from a Dammar gum, a plasticizer, and an elastomer. Elastomers, commonly described as rubbery materials, differ from plasticizers in that they have a great capacity for large elastic deformation under an applied stress.

In some aspects, the composition comprises a compound derived from a Dammar gum and an elastomer.

In some aspects, the elastomer is an elastomer gel from epoxidized vegetable oil. U.S. Appl. Publication No. 2022/0194912 describes the preparation of an elastomer comprising the reaction product of at least one epoxidized molecule and at least one crosslinker.

In some aspects, the elastomer comprises the reaction product of:
(a) at least one compound of formula (I)

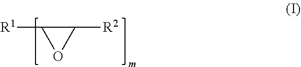

wherein
R¹ is C₆-C_{100,000} alkyl group, C₆-C_{100,000} heteroalkyl group, C₆-C_{100,000} alkene group, C₆-C_{100,000} heteroalkene group, C₆-C_{100,000} alkyne group, C₆-C_{100,000} heteroalkyne group, C₆-C_{100,000} cyclic group, or C₆-C_{100,000} heterocyclic group;
R² is hydrogen, C₁-C_{100,000} alkyl group, C₁-C_{100,000} heteroalkyl group, C₂-C_{100,000} alkene group, C₂-C_{100,000} heteroalkene group, C₂-C_{100,000} alkyne group, C₂-C_{100,000} heteroalkyne group, C₃-C_{100,000} cyclic group, or C₃-C_{100,000} heterocyclic group; and
m is an integer from 2 to 1,000; and
(b) at least one crosslinker selected from the group consisting of:
(1) a carboxylic acid comprising the structure of formula (II)

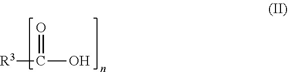

wherein
R³ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
n is an integer from 2 to 10;
(2) an anhydride comprising the structure of formula (III)

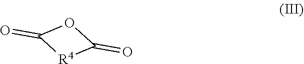

wherein
R⁴ is C₂-C₂₀₀ alkyl group, C₂-C₂₀₀ heteroalkyl group, C₂-C₂₀₀ alkene group, C₂-cyclic group, or C₂-C₂₀₀ heterocyclic group;
an amine comprising the structure of formula (IV)

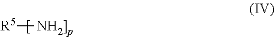

wherein
R⁵ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
p is an integer from 2 to 10;
an alcohol comprising the structure of formula (V)

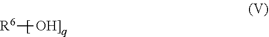

wherein
R⁶ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
q is an integer from 2 to 10;
a hydroxyl carboxylic acid comprising the structure of formula (VI)

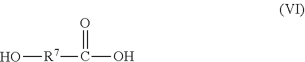

wherein
R⁷ is C₁-C₂₀₀ alkyl group, C₁-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
a amine carboxylic acid comprising the structure of formula (VII)

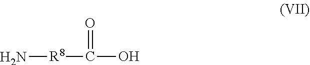

wherein
R⁸ is C₁-C₂₀₀ alkyl group, C₁-C_{100,000} heteroalkyl group, C₂-C₂₀₀ alkene group, C₂-cyclic group, or C₂-C₂₀₀ heterocyclic group;
and combinations thereof.

In some aspects, the elastomer is a crosslinked polyester elastomer. U.S. Appl. Publication No. 2022/0195178 describes the preparation of a crosslinked polyester elastomer comprising the reaction product of (i) at least one polycarboxylic acid, at least one polycarboxylic ester, or a combination thereof; and (ii) at least one polyol.

In some aspects, the elastomer comprises the reaction product of:
(i) at least one polycarboxylic acid of formula (IA)

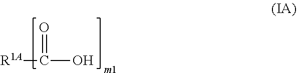

wherein
R^{1A} is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
m1 is an integer from 2 to 10; or
(ii) at least one polycarboxylic acid ester of formula (IIA)

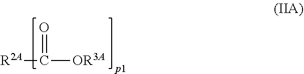

wherein
R^{2A} is C₁-C₂₂ alkyl group, C₂-C₂₂ alkene group, or C₃-C₂₂ cyclic group;
R^{3A} is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
p1 is an integer from 3 to 10; or
(iii) a combination thereof; and
(iv) at least one polyol of formula (IIIA)

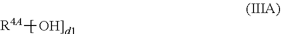

wherein
R^{4A} is C₂-C₂₀₀ alkyl group, C₂-C₂₀₀ heteroalkyl group, C₂-C₂₀₀ alkene group, C₂-cyclic group, or C₂-C₂₀₀ heterocyclic group; and
d1 is an integer from 2 to 10.

In some aspects, the elastomer is a crosslinked polyester elastomer. U.S. Appl. No. 63/492,429 describes the preparation of a crosslinked polyester elastomer comprising the reaction product of (i) at least one di-carboxylic acid or tri-carboxylic acid; (ii) at least one mono-carboxylic acid; and (ii) at least one polyol.

In some aspects, the elastomer comprises the reaction product of:
(i) at least one di-carboxylic acid is a compound of formula (IB)

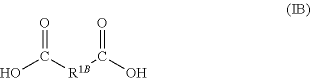

wherein
R^{1B} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group; or
(ii) at least one tri-carboxylic acid is a compound of formula (IIB)

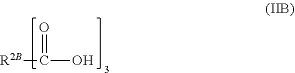

wherein
R^{2B} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group; and
(iii) at least one mono-carboxylic acid is a compound of formula (IIIB)

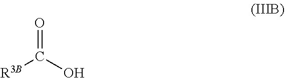

wherein
R^{3B} is C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group; and
(iv) at least one polyol is a compound of formula (IVB)

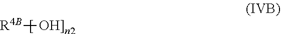

wherein
R^{4B} is C₃-C₅₀ alkyl group, C₃-C₅₀ heteroalkyl group, C₃-C₅₀ alkene group, C₃-C₅₀ heteroalkene group, C₃-C₅₀ cyclic group, or C₃-C₅₀ heterocyclic group; and
n2 is an integer from 2 to 10.

In some aspects, the elastomer is a crosslinked polyester elastomer. U.S. Appl. No. 63/492,305 describes the preparation of a crosslinked polyester elastomer comprising the reaction product of (i) at least one activated polycarboxylic acid; and (ii) at least one polyol.

In some aspects, the elastomer comprises the reaction product of:
(i) at least one activated polycarboxylic acid is a compound of formula (IC)

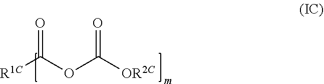

wherein
R^{1C} is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
R^{2C} is a C₁-C₆₀ monovalent hydrocarbon group; and
m is an integer from 2 to 10; and
(ii) the at least one polyol is a compound of formula (IIC)

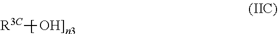

wherein
R^{3C} is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
n3 is an integer from 2 to 10.

In some aspects, the elastomer is a siloxane elastomer.

In some aspects, the compositions comprise an elastomer in a weight ratio from about 0.01% to about 80% of the total weight of the composition. In some aspects, the compositions comprises an elastomer in a weight ratio from about 0.01% to about 80%, from about 0.01% to about 60%, from about 0.01% to about 40%, from about 0.01% to about 20%, from about 0.01% to about 10%, from about 0.01% to about 5%, from about 0.01% to about 1%, from about 1% to about 80%, from about 1% to about 60%, from about 1% to about 40%, from about 1% to about 20%, from about 1% to about 10%, from about 1% to about 5%, from about 5% to about 80%, from about 5% to about 60%, from about 5% to about 40%, from about 5% to about 20%, from about 5% to about 10%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% of the total weight of the composition. In some aspects, the compositions comprise an elastomer in a weight ratio of about 0.01%, about 1%, about 5%, about 10%, about 20%, about 40%, about 60%, or about 80% of the total weight of the composition.

The compositions comprising a compound derived from a Dammar gum can further comprise at least one additional component selected from the group consisting of water, an aqueous solvent (e.g. alcohol or other water compatible solvent), a non-aqueous solvent, emollients, humectants, oils (polar and non-polar), conditioning agents, emulsifiers, surfactants, thickeners, stiffening agents, medicaments, fragrances, preservatives, deodorant components, anti-perspirant compounds, skin protecting agents, pigments, sunscreens and mixtures thereof, among others, wherein said composition, when deposited onto a surface, dries (after evaporation of solvent and water) into a uniform, flexible, durable film exhibiting water resistance, flexibility, durability and optionally, transfer resistance when the film also comprises a pigment, dye, oil and/or active component.

In some aspects, the additional component(s) comprise from about 0.01% to about 80%, from about 0.01% to about 60%, from about 0.01% to about 40%, from about 0.01% to about 20%, from about 0.01% to about 10%, from about 0.01% to about 5%, from about 0.01% to about 1%, from about 1% to about 80%, from about 1% to about 60%, from about 1% to about 40%, from about 1% to about 20%, from about 1% to about 10%, from about 1% to about 5%, from about 5% to about 80%, from about 5% to about 60%, from about 5% to about 40%, from about 5% to about 20%, from about 5% to about 10%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% of the total weight of the composition. In some aspects, the compositions comprise the additional component(s) in a weight ratio of about 0.01%, about 1%, about 5%, about 10%, about 20%, about 40%, about 60%, or about 80% of the total weight of the composition.

### V. Compound Derived from Dammar Gum Prepared by Process

The present disclosure provides a compound derived from Dammar gum prepared by a process comprising:
(i) dissolving Dammar gum in a first solvent;
(ii) filtering the solution formed in (i);
(iii) concentrating the filtrate from (ii);
(iv) precipitating the concentrate with a second solvent;
(v) isolating the compound derived from Dammar gum.

### VI. Methods of Making a Compound Derived from Dammar Gum

The present disclosure provides a method of making a compound derived from a Dammar gum comprising:
(i) dissolving a Dammar gum in a first solvent;
(ii) filtering the solution of (i);
(iii) concentrating the filtrate of (ii);
(iv) precipitating the concentrate with a second solvent; and
(v) isolating the compound derived from Dammar gum.

The present disclosure provides a method of making a compound derived from a Dammar gum comprising:
(i) dissolving a Dammar gum in a first solvent;
(ii) filtering the solution of (i);
(iii) concentrating the filtrate of (ii);
(iv) precipitating the concentrate with an alcohol; and
(v) isolating the compound derived from Dammar gum.

In some aspects, the Dammar gum is a natural Dammar gum. In some aspects, the Dammar gum is a commercially available Dammar gum. In some aspects, the Dammar gum is Dammar gum resin from Indonesia that includes tree bark. In some aspects, the Dammar gum is DAMAR-EZ Organic (Nexira, Rouen, France) that has the tree bark removed. In some aspects, the Dammar gum can be washed with water. In some aspects, the Dammar gum can be ground. In some aspects, the Dammar gum can ground to a fine powder. In some aspects, the Dammar gum can be in lumps.

In some aspects, the first solvent is a polar aprotic solvent. In some aspects, the first solvent is a polar aprotic solvent selected from the group consisting of acetonitrile, dichloromethane, tetrahydrofuran, ethyl acetate, dimethylformamide, dimethyl sulfoxide, acetone, N-methylpyrrolidone, and combinations thereof.

Is some aspects, the first solvent is a volatile hydrocarbon. In some aspects, the volatile hydrocarbon is an aliphatic hydrocarbon, a straight chain hydrocarbon, a branched chain hydrocarbon, or a combination thereof. In some aspects, the volatile hydrocarbon is a branched chain hydrocarbon, such as a C₁₂-C₁₄ isoparaffin. In some aspects, the volatile hydrocarbon is isododecane.

In some aspects, the first solvent is selected from the group consisting of acetonitrile, dichloromethane, tetrahydrofuran, ethyl acetate, dimethylformamide, dimethyl sulfoxide, acetone, N-methylpyrrolidone, dipentene, toluene, dichloromethane, chloroform, tetrachloromethane, isododecane, and combinations thereof. In some aspects, the first solvent is selected from the group consisting of dipentene, toluene, dichloromethane, chloroform, tetrachloromethane, acetone, isododecane, and combinations thereof.

In some aspects, the first solvent is toluene. In some aspects, the first solvent is dipentene. In some aspects, the first solvent is acetone. In some aspects, the first solvent is a combination of acetone and isododecane.

In some aspects, the first solvent is a ratio of 1:2 acetone to isododecane by weight.

In some aspects, the dissolving is at a temperature from about 20° C. to about 300° C. In some aspects, the dissolving is at a temperature from about 20° C. to about 300° C., from about 20° C. to about 250° C., from about 20° C. to about 200° C., from about 20° C. to about 150° C., from about 20° C. to about 100° C., from about 20° C. to about 50° C., from about 50° C. to about 300° C. In some aspects, the dissolving is at a temperature from about 20° C. to about 300° C., from about 50° C. to about 250° C., from about 50° C. to about 200° C., from about 50° C. to about 150° C., from about 50° C. to about 100° C., from about 100° C. to about 300° C., from about 100° C. to about 250° C., from about 100° C. to about 200° C., from about 100° C. to about 150° C., from about 150° C. to about 300° C., from about 150° C. to about 250° C., from about 150° C. to about 200° C., from about 200° C. to about 300° C., from about 200° C. to about 250° C., or from about 250° C. to about 300° C. In some aspects, the dissolving is at a temperature from about 20° C. to about 200° C. In some aspects, the dissolving is at a temperature from about 20° C. to about 150° C. In some aspects, the dissolving is at a temperature from about 20° C. to about 100° C. In some aspects, the dissolving is at a temperature from about 50° C. to about 200° C. In some aspects, the dissolving is at a temperature from about 50° C. to about 150° C. In some aspects, the dissolving is at a temperature from about 50° C. to about 100° C.

In some aspects, the dissolving is for a time from about 1 minute to about 120 minutes, from about 1 minute to about 90 minutes, from about 1 minute to about 60 minutes, from about 1 minute to about 30 minutes, from about 1 minute to about 15 minutes, from about 1 minute to about 5 minutes, from about 5 minutes to about 120 minutes, from about 5 minutes to about 90 minutes, from about 5 minutes to about 60 minutes, from about 5 minutes to about 30 minutes, from about 5 minutes to about 15 minutes, from about 15 minutes to about 120 minutes, from about 15 minutes to about 90 minutes, from about 15 minutes to about 60 minutes, from about 15 minutes to about 30 minutes, from about 30 minutes to about 90 minutes, from about 30 minutes to about 60 minutes, or from about 60 minutes to about 120 minutes. In some aspects, the dissolving is for a time of about 1 minute, about 5 minutes, about 15 minutes, about 30 minutes, about 60 minutes, or about 120 minutes.

In some aspects, the dissolved solution from (i) is filtered using glass wool. In some aspects, the dissolved solution from (i) is filtered using vacuum filtration. In some aspects, the dissolved solution from (i) is cooled to room temperature before filtration. In some aspects, the dissolved solution from (i) is not allowed to cool before filtration.

In some aspects, the second solvent is selected from the group consisting of an alcohol, acetone, or combinations thereof. In some aspects the second solvent is an alcohol selected from the group consisting of ethanol, methanol, isopropyl alcohol, or combinations thereof.

In some aspects, the second solvent is ethanol. In some aspects, the second solvent is methanol. In some aspects, the second solvent is isopropyl alcohol. In some aspects, the second solvent is a combination of ethanol, methanol, and isopropyl alcohol. In some aspects the second solvent is a combination of 80-95% ethanol, 1-10% methanol, and 1-10% isopropyl alcohol. In some aspects, the second solvent is a combination of 88-91% ethanol, 4-5% methanol, and 4.5-5.5% isopropyl alcohol. In some aspects, the second solvent is acetone.

### VII. Personal Care Formulations

In some aspects, the present disclosure is directed to a personal care formulation comprising the compound or the composition.

In some aspects, said formulation forms a film when applied to skin or lips.

In some aspects, the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a lip gloss, a lip color, a liquid lip, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

In some aspects, the personal care formulation is a lipstick.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Method of Making the Compound Derived from Ground Dammar Gum

Natural Dammar gum was ground or was purchased in ground form. The ground Dammar gum was dissolved in a solvent at a temperature from about 20° C. to about 300° C., depending upon to the boiling point of the solvent used. Solvents tested include dipentene, toluene, dichloromethane, chloroform, and tetrachloromethane, acetone, isododecane, and combinations thereof. The solution was filtered under pressure and concentrated. In some tests, the solution was filtered through glass wool. The filtrate was precipitated from the concentrate by the addition of a solvent. Solvents tested include ethanol, methanol, isopropyl alcohol, acetone, and combinations thereof. In some tests, a minimal amount of solvent was used. The precipitate was isolated by vacuum filtration, washed with a solvent, and dried to provide the compound derived from Dammar gum.

The compound derived from Dammar gum was characterized by ¹C NMR as comprising at least 80 mol % saturated branched hydrocarbons; between 5 mol % and 20 mol % unsaturated hydrocarbons; and less than 1 mol % of carbonyl carbons, wherein the carbonyl carbons comprise aldehydes, esters, and acids. The 1H NMR of the compound derived from Dammar gum (solvent=CDCl₃) is shown in TABLE 1 and the 1C NMR of the compound derived from Dammar gum (solvent=Cr(AcAc)₃ in CDCl₃) is shown in TABLE 2.

TABLE 1
¹H NMR Data for Compound Derived from Dammar gum

| δ (ppm) | Tentative assignment | Relative molar ratio |
|---|---|---|
| 0.89 to 2.10 | Highly branched hydrocarbons | 45.66 |
| | —(CH₂)ₙ- and CH/CH₃ | |
| 4.85 to 3.23 | Unassigned | 0.43 |
| 5.78 to 4.67 | Unsaturation | 2.00 |
| 7.17 | Unsaturation, aromatic | 0.01 |
| 9.40 | Unassigned | 0.03 |
| 9.33 | Unassigned | 0.04 |

H_{olefin}:H_{total other} = 1.00:23.00

TABLE 2
¹C NMR Data for Compound Derived from Dammar gum

| δ (ppm) | Tentative assignment | Relative molar ratio |
|---|---|---|
| 55.5 to 12.4 | Highly branched | 86.34 |
| | hydrocarbons (CH) | |
| 75.6 to 75.0 | —CHO— | 0.52 |
| 108.7 to 107.0 | CH₂ unsaturation | trace |
| 113.1 | CH₂ unsaturation | 0.17 |
| 134.8 to 124.3 | CH carbon of unsaturation | 6.29 |
| 137.6 to 135.0 | Quaternary carbon of unsaturation | 5.66 |
| 147.0 | Unsaturation | 0.13 |
| 152.2 | —C═C < unsaturation | 0.05 |
| 178.5 | Ester/acid type carbonyl carbon | 0.15 |
| 206.8 | Aldehyde type carbonyl carbon | 0.16 |
| 217.6 to 217.2 | unassigned | 0.52 |

C_{olefin}:C_{total other} = 1.00:7.13

The isolated compound derived from Dammar gum was characterized by GPC using Cirrus GPC version 3.4.2 in manual peak selection mode with a sample concentration of 1 mg/mL and an injection volume of 10 μL. The following results were obtained based on the columns and solvents listed:
(a) as having a Mn of about 3153 g/mol and a polydispersity of about 1.5 using polystyrene 500-1,000,000 Da standards, in methylene chloride solvent, with two PHENOGEL (Phenomenex, Torrance, CA) 5 μm linear (2) columns (dimensions: 300 mm×7.8 mm) and one PHENOGEL 5 μm linear (2) guard column (dimensions: 50 mm×7.8 mm); or
(b) as having a Mn of about 3418 g/mol and a polydispersity of about 1.3 using polystyrene 500-1,000,000 Da standards, in methylene chloride solvent, with two PLgel (Phenomenex, Torrance, CA) 5 μm MIXED-D columns (dimensions: 300 mm×7.5 mm), and one PLgel 5 μm MIXED guard column (dimensions: 50 mm×7.8 mm); or
(c) as having a Mn of about 612 and a polydispersity of about 5.7 using polystyrene 500-1,000,000 Da standards, in toluene solvent, with two PHENOGEL 5 μm linear (2) columns (dimensions: 300 mm×7.8 mm) and one PHENOGEL 5 μm linear (2) guard column (dimensions: 50 mm×7.8 mm).

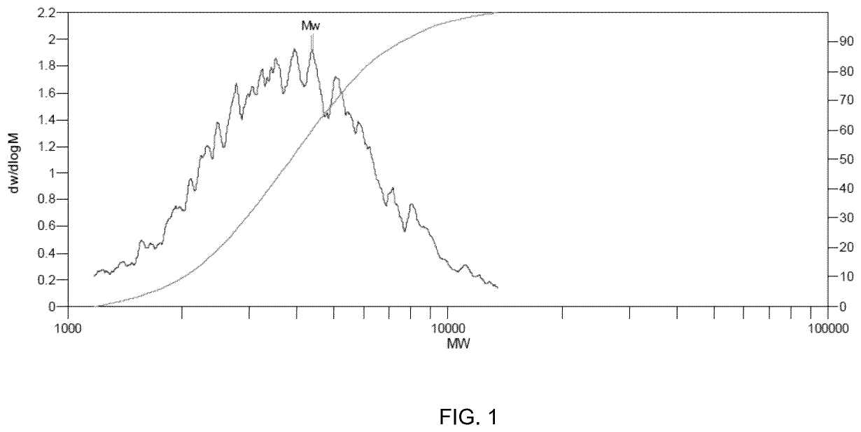

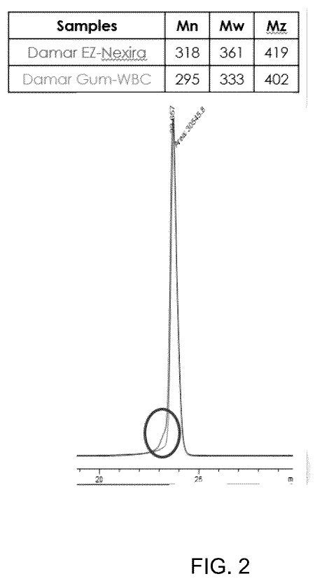

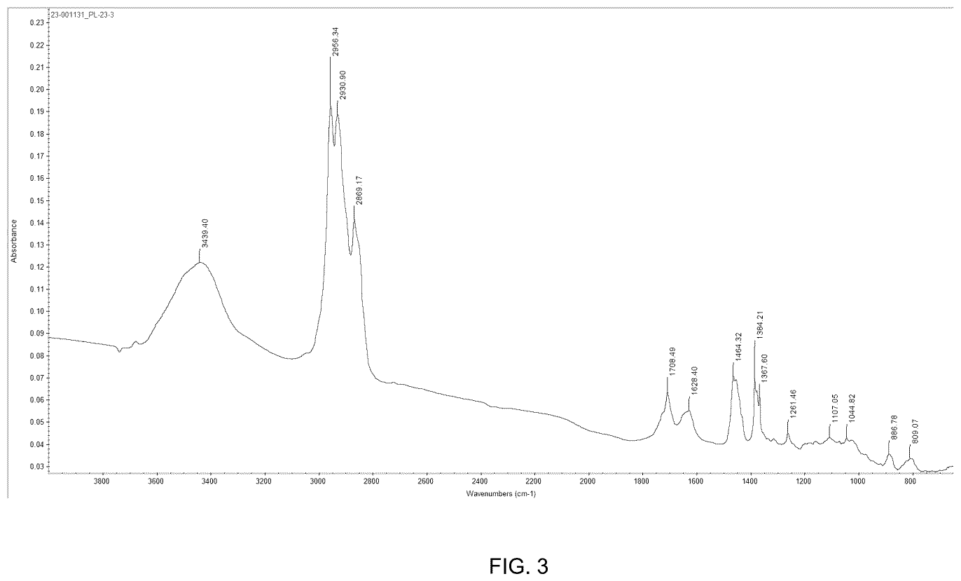

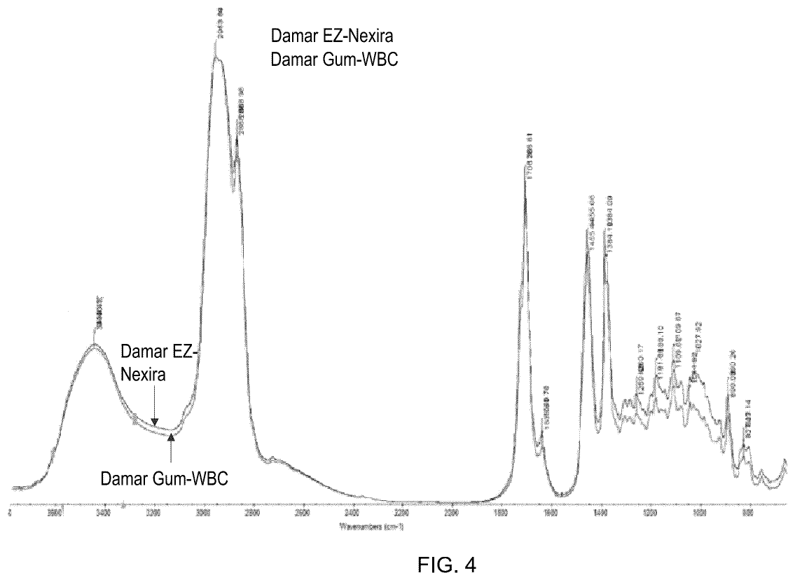

A GPC of the compound derived from Dammar gum is provided in FIG. 1. A GPC of the natural Dammar gum is provide in FIG. 2. Comparison of the GPC in FIGS. 1 and 2 shows that the Mw and Mn of the compound derived from Dammar gum is different from that obtained from the natural Dammar gums.

The isolated compound derived from Dammar gum was characterized by TGA using a TA Instruments Q50 V20 Build 39 (TA Instruments, New Castle, DE) with a temperature ramp of 10° C./minute using Universal V4.5A software. The compound had a highest rate of decomposition of about 395° C. as measured by thermal gravimetric analysis (TGA).

The glass transition temperature (T_{g}) of the isolated compound derived from Dammar gum was characterized using TA Instruments Discovery DSC 250 (TA Instruments, New Castle, DE) with a differential scanning calorimeter (DSC) analysis with differential heat flow measurement using midpoint transition analysis. The compound had a glass transition temperature (T_{g}) of 54.69° C. (run 1) and 64.54° C. (run 2).

### Example 2: Method of Making the Compound Derived from a Dammar Gum with Precipitation from a Polar Aprotic Solvent

The method of Example 1 was used to prepare the Dammar gum. Raw Dammar gum lumps (36 grams) were dissolved in a mixture of a polar aprotic solvent and a volatile hydrocarbon (3.6 grams/7.2 grams) heated to reflux. The solution was filtered using a pressure filter with glass wool to remove tree bark. A polar aprotic solvent (40 grams) was added to the solution in order to precipitate the product. The solid product was removed via filtration and washed with a polar aprotic solvent (5 mL). The solid product was dried, resulting in 5.61 grams of product with a yield of 15.6%).

### Example 3: Method of Making the Compound Derived from a Dammar Gum with Precipitation from a Polar Aprotic Solvent

The method of Example 1 was used to prepare the Dammar gum. Raw Dammar gum lumps (18 grams) were dissolved in mixture of a polar aprotic solvent and a volatile hydrocarbon (1.8 grams/3.6 grams) heated to reflux. The solution was filtered using a pressure filter with glass wool to remove tree bark. A polar aprotic solvent (60 grams) was added to the solution in order to precipitate the compound. The compound was removed via filtration and washed with a polar aprotic solvent (5 mL). The compound was dried, resulting in 3.40 grams of product with a yield of 18.9%.

### Example 4: Method of Making the Compound Derived from Dammar Gum with Precipitation from a Polar Aprotic Solvent

The method of Example 1 was used to prepare the Dammar gum. The Dammar gum (30 grams) was ground into a powder. The ground Dammar gum powder was then mixed with a polar aprotic solvent (80 grams) in a flask. The mixture formed was agitated and heated to reflux for about 30 minutes. The mixture was allowed to cool to room temperature. The mixture was filtered and the solid was washed with a polar aprotic solvent (5 mL). The compound was dried, resulting in 4.55 grams of product with a yield=15.2%.

### Example 5: Preparation and Transfer Resistance In Vitro Test of Cosmetic Formulations

Cosmetic formulations using compounds derived from Dammar gum were prepared according to TABLE 3. Compound 1 Derived from Dammar Gum (TEST 2) and Compound 2 Derived from Dammar Gum (TEST 7) were prepared using the method of Example 1.

TABLE 3
Cosmetic Formulations Prepared and Tested for Transfer Resistance

| | TEST 1 | TEST 2 | TEST 3 | TEST 4 | TEST 5 | TEST 6 | TEST 7 |
|---|---|---|---|---|---|---|---|
| BENTONE | 19.05 | 19.05 | 19.05 | 19.05 | 19.05 | 19.05 | 19.05 |
| GEL ISD V¹ | | | | | | | |
| Red Lake 7 | 7.5 | 7.5 | 7.5 | 7.5 | 7.5 | 7.5 | 7.5 |
| TiO₂ (MT- | 1.47 | 1.47 | 1.47 | 1.47 | 1.47 | 1.47 | 1.47 |
| 100TV²) | | | | | | | |
| Isododecane | 21.98 | 16.98 | 9.47 | 16.98 | 16.98 | 16.98 | 16.98 |
| SE 30³ | | | 5.63 | 2.25 | | | |
| SR1000⁴ | | | 6.88 | 2.75 | | | |
| Compound 1 | | 5 | | | | | |
| Derived From | | | | | | | |
| Dammar Gum | | | | | | | |
| Ground | | | | | 5 | | |
| Dammar Gum | | | | | | | |
| Indo⁵ | | | | | | | |
| Water | | | | | | 5 | |
| Washed⁶ | | | | | | | |
| DAMAR-EZ | | | | | | | |
| Organic⁷ | | | | | | | |
| Compound 2 | | | | | | | 5 |
| Derived from | | | | | | | |
| Dammar Gum | | | | | | | |

¹BENTONE GEL ISD V (Elementis Specialties, East Windsor, NJ).
²Micro Titanium Dioxide (MT-100TV (Tayca Corporation, Osaka, Japan))
³Dimethicone gum (Silsoft SE 30 (Momentive Performance Materials, Inc., Niskayuna, New York)
⁴SR1000 Resin ((Momentive Performance Materials, Inc., Niskayuna, New York)
⁵Ground Dammar Gum Indo (CV Lafasa Agrochem, Indonesia) is a commercially available form of the natural gum which was ground to a fine powder before use.
⁶Ground Dammar Gum Indo that was washed with water before use.
⁷DAMAR-EZ Organic (Nexira, Rouen, France) is a commercially available compound that has the tree bark removed.

### Example 6: Transfer Resistance In Vitro Test of Cosmetic Formulations

The formulations prepared in Example 5 were subject to the following transfer resistance in-vitro test protocol:

(a) Transfer resistance: VITRO-SKIN (IMS Testing Group, Bunnell, FL) was hydrated using 140 grams of distilled water and 60 grams of glycerin. Once hydrated overnight, it was placed in the center of a cutout of a previously prepared printing stencil, with the rough side to the top, then flattened against the bottom piece of sheeting to remove entrapped air and wrinkles. Then a sample from the tested formulation was placed in an approximately 0.5 mm thick line along the left side of the VITRO-SKIN. Using the 1 mm gap side of the wet film applicator, a uniform coating of sample over the entire surface of the VITRO-SKIN was achieved by slowly pulling the wet film applicator over the flat VITRO-SKIN. Then, the so-coated VITRO-SKIN was placed in a 50° C. oven for 30 minutes, after which it was placed on a clean clipboard. Using a rubber band, a piece of transfer cloth was tightly attached to the bottom of the 500 gm weight. The weight was centered on the coated piece of VITRO-SKIN, which was securely held in place while the weight was rotated in small steps through 360° by moving it through as many partial rotations as needed to make an entire circle. Then the cotton cloth was removed from the weight and visually examined for the degree of color transfer.

(b) Water resistance: The same process as in (a) was employed, except that prior to the cloth-covered weight application, 9 drops of distilled water were applied onto the center of the coated and dried VITRO-SKIN.

(c) Oil resistance: Same procedure as (b) but instead of water, avocado oil was used.

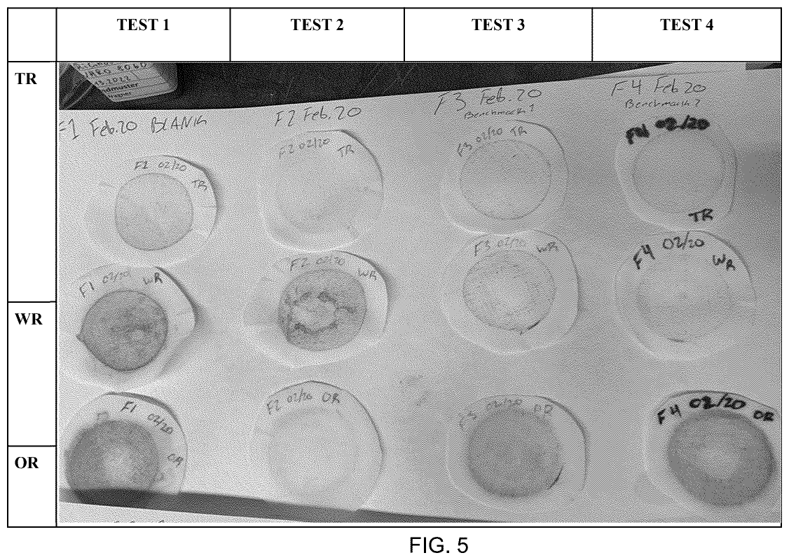

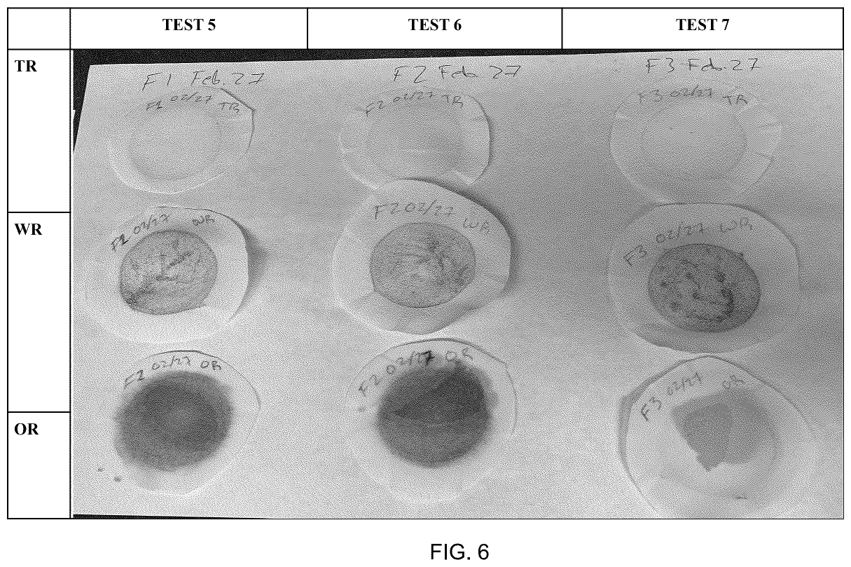

The results are depicted in FIGS. 5 and 6, where a lighter color indicates less color transfer from the VITRO-SKIN to the cotton cloth. As shown in the FIGs, Compound 1 derived from Dammar gum (TEST 2) and Compound 2 derived from Dammar gum (TEST 7) showed good transfer resistance, water resistance, and oil resistance. In particular, the transfer resistance and oil resistance tests for Compound 1 derived from Dammar gum (TEST 2) showed very little transference. Similarly, the transfer resistance and oil resistance tests for Compound 1 derived from Dammar gum (TEST 7) showed very little transference.

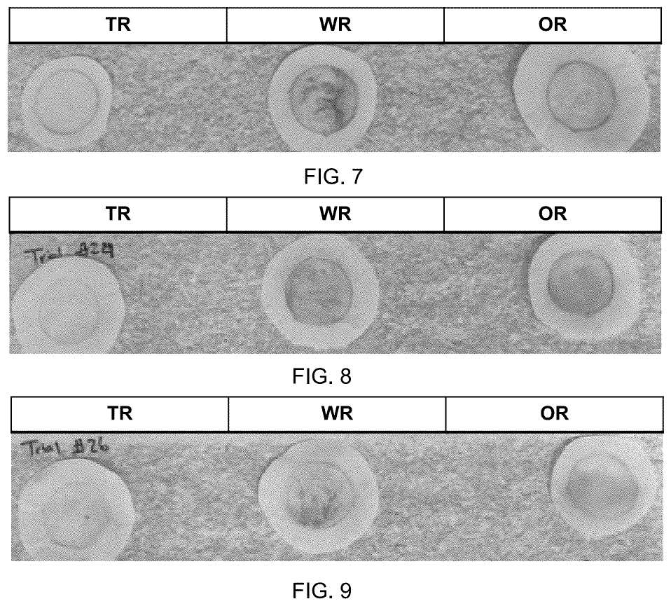

The compounds derived from Dammar gum using the methods of Examples 2-4 were also tested for transfer resistance, water resistance, and oil resistance with results shown in FIGS. 7-9.

### Other Aspects

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

1. A compound derived from a Dammar gum comprising: at least about 80 mol % saturated branched hydrocarbons; between about 5 mol % and about 20 mol % unsaturated hydrocarbons; and less than about 1 mol % of carbonyl carbons, wherein the carbonyl carbons comprise aldehydes, esters, and acids.

2. The compound of claim 1, further characterized as having an average number molecular weight (Mn) of about 2000 g/mol to about 5000 g/mol as measured by gel permeation chromatography (GPC).

3. The compound of claim 1, further characterized as having a Mn of about 2500 g/mol to about 3700 g/mol as measured by GPC.

4. The compound of claim 1, further characterized as having a Mn of about 3418 g/mol as measured by GPC.

5. The compound of claim 1, further characterized by GPC:
(a) as having a Mn of about 3153 g/mol and a polydispersity of about 1.5 using polystyrene 500-1,000,000 Da standards, in methylene chloride solvent, with two PHENOGEL 5 μm linear (2) columns (dimensions: 300 mm×7.8 mm) and one PHENOGEL 5 μm linear (2) guard column (dimensions: 50 mm×7.8 mm); or
(b) as having a Mn of about 3418 g/mol and a polydispersity of about 1.3 using polystyrene 500-1,000,000 Da standards, in methylene chloride solvent, with two PLgel 5 μm MIXED-D columns (dimensions: 300 mm×7.5 mm), and one PLgel 5 μm MIXED guard column (dimensions: 50 mm×7.8 mm); or
(c) as having a Mn of about 612 and a polydispersity of about 5.7 using polystyrene 500-1,000,000 Da standards, in toluene solvent, with two PHENOGEL 5 μm linear (2) columns (dimensions: 300 mm×7.8 mm) and one PHENOGEL 5 μm linear (2) guard column (dimensions: 50 mm×7.8 mm).

6. The compound of claim 1, further characterized as having a highest rate of decomposition of about 350° C. to about 450° C. as measured by thermal gravimetric analysis (TGA).

7. The compound of claim 1, further characterized as having a highest rate of decomposition of about 395° C. as determined by TGA.

8. The compound of claim 1, further characterized as having a IR spectrum with peaks at 3439, 2956, 2931, and 2869 cm⁻¹ (±4 cm⁻¹).

9. The compound of claim 1, further characterized as having a IR spectrum with peaks at 3439, 2956, 2931, 2869, 1708, 1628, 1464, and 1384 cm⁻¹ (±4 cm⁻¹).

10. A composition comprising the compound of claim 1 and further comprising:
(a) at least one non-volatile oil; or
(b) at least one volatile oil; or
(c) at least one elastomer; or
a combination thereof.

11. The composition of claim 10, wherein the at least one non-volatile oil is a plasticizer.

12. The composition of claim 11, wherein the plasticizer is selected from the group consisting of a trimethylpentanediol/adipic acid/glycerin crosspolymer, capryloyl glycerin/sebacic acid copolymer, Brassica campestris/Aleurites fordii oil copolymer, PPG-12/SMDI copolymer, polyisobutylene, polyisobutene 1200, isocetyl salicylate, polyhydroxystearic acid, and dicaprylyl/capryl sebacate.

13. The composition of claim 11, wherein the plasticizer is selected from the group consisting of LEXOREZ 200 MB, VELLAPLEX MB, GLOSSAMER L6600, ADEKA NOL OU-1, LIPFEEL Natural MB, polyisobutylene, polyisobutene 1200, DERMOL ICSA, KESTER WAX K-60P, and HALLGREEN CCS.

14. The composition of claim 10, wherein the composition does not comprise a volatile alcohol.

15. A personal care formulation comprising the compound of claim 1.

16. The personal care formulation of claim 15, wherein said formulation forms a film when applied to skin or lips.

17. The personal care formulation of claim 15, wherein the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a lip gloss, a lip color, a liquid lip, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

18. A personal care formulation comprising the composition of claim 10.

19. The personal care formulation of claim 18, wherein the personal care formulation is selected from the group consisting of a deodorant, an antiperspirant, a skin cream, a facial cream, a hair shampoo, a hair conditioner, a mousse, a hair styling gel, a hair spray, a protective cream, a lipstick, a lip gloss, a lip color, a liquid lip, a facial foundation, blushes, makeup, a mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.
